(19) United States
(12) Patent Application Publication
(10) Pub. No.: US 2022/0288095 A1
(43) Pub. Date: Sep. 15, 2022

(54) SIALIC ACID FOR USE IN THE TREATMENT OF PSORIASIS

(71) Applicant: LifeScience AS, Orsta (NO)

(72) Inventor: Jan Remmereit, Hovdebygda (NO)

(21) Appl. No.: 17/629,934

(22) PCT Filed: Jul. 24, 2020
(86) PCT No.: PCT/IB2020/000616

§ 371 Date: Jan. 25, 2022

Related U.S. Application Data
(60) Provisional application No. 62/879,202, filed on Jul. 26, 2019.

(51) Int. Cl. A61K 31/7012; A61K 31/202; A61P 17/06

(52) U.S. Cl. CPC A61K 31/7012; A61K 31/202; A61P 17/06

(57) ABSTRACT

The present invention relates to compositions and methods for treating psoriasis, and in particular to compositions and methods that utilize sialic acids or sialic acid-containing compounds such as gangliosides to treat psoriasis.

### FIELD OF THE INVENTION

The present invention relates to compositions and methods for treating psoriasis, and in particular to compositions and methods that utilize sialic acids or sialic acid-containing compounds such as gangliosides to treat psoriasis.

### BACKGROUND OF THE INVENTION

Psoriasis is a common skin condition that speeds up the life cycle of skin cells. It causes cells to build up rapidly on the surface of the skin. The extra skin cells form scales and red patches that are itchy and sometimes painful. Psoriasis is a chronic disease that often comes and goes. The main goal of treatment is to stop the skin cells from growing so quickly. There is no cure for psoriasis, but symptoms can be managed in some cases.

Psoriasis signs and symptoms can vary. Common signs and symptoms include: Red patches of skin covered with thick, silvery scales; Small scaling spots (commonly seen in children); Dry, cracked skin that may bleed; Itching, burning or soreness; Thickened, pitted or ridged nails; and Swollen and stiff joints. Psoriasis patches can range from a few spots of dandruff-like scaling to major eruptions that cover large areas.

Most types of psoriasis go through cycles, flaring for a few weeks or months, then subsiding for a time or even going into complete remission. There are several types of psoriasis.

Plaque psoriasis. The most common form, plaque psoriasis causes dry, raised, red skin lesions (plaques) covered with silvery scales. The plaques might be itchy or painful and there may be few or many. They can occur anywhere on your body, including your genitals and the soft tissue inside your mouth.

Nail psoriasis. Psoriasis can affect fingernails and toenails, causing pitting, abnormal nail growth and discoloration. Psoriatic nails might loosen and separate from the nail bed (onycholysis). Severe cases may cause the nail to crumble.

Guttate psoriasis. This type primarily affects young adults and children. It's usually triggered by a bacterial infection such as strep throat. It's marked by small, water-drop-shaped, scaling lesions on your trunk, arms, legs and scalp. The lesions are covered by a fine scale and aren't as thick as typical plaques are.

Inverse psoriasis. This mainly affects the skin in the armpits, in the groin, under the breasts and around the genitals. Inverse psoriasis causes smooth patches of red, inflamed skin that worsen with friction and sweating. Fungal infections may trigger this type of psoriasis.

Pustular psoriasis. This uncommon form of psoriasis can occur in widespread patches (generalized pustular psoriasis) or in smaller areas on your hands, feet or fingertips. It generally develops quickly, with pus-filled blisters appearing just hours after your skin becomes red and tender. The blisters may come and go frequently. Generalized pustular psoriasis can also cause fever, chills, severe itching and diarrhea.

Erythrodermic psoriasis. The least common type of psoriasis, erythrodermic psoriasis can cover your entire body with a red, peeling rash that can itch or burn intensely.

Psoriatic arthritis. In addition to inflamed, scaly skin, psoriatic arthritis causes swollen, painful joints that are typical of arthritis. Sometimes the joint symptoms are the first or only manifestation of psoriasis or at times only nail changes are seen. Symptoms range from mild to severe, and psoriatic arthritis can affect any joint. Although the disease usually isn't as crippling as other forms of arthritis, it can cause stiffness and progressive joint damage that in the most serious cases may lead to permanent deformity.

### SUMMARY OF THE INVENTION

In some preferred embodiments, the present invention provides a composition comprising an effective amount of an active agent selected from the group consisting of a sialic acid, a sialic acid precursor and a sialic acid containing compound for use in treating or prophylaxis of psoriasis. In other preferred embodiments, the present invention provides methods of treating or providing prophylaxis of psoriasis comprising administering to a subject in need thereof an effective amount of an active agent selected from the group consisting of a sialic acid, a sialic acid precursor and a sialic acid containing compound.

In some preferred embodiments, the sialic acid is selected from the group consisting of n-glycolylneuraminic acid (NGNA), n-acetylneuraminic acid (NANA), N-Acetyl-D-mannosamine, neuraminic acid, 5-N-Acetyl-4-O-acetyl-neuraminic acid, 5-N-Acetyl-7-O-acetyl-neuraminic acid, 5-N-Acetyl-8-O-acetyl-neuraminic acid, 5-N-Acetyl-9-O-acetyl-neuraminic acid, 5-N-Acetyl-4,9-di-O-acetyl-neuraminic acid, 5-N-Acetyl-7,9-di-O-acetyl-neuraminic acid, 5-N-Acetyl-8,9-di-O-acetyl-neuraminic acid, 5-N-Acetyl-7,8,9-tri-O-acetyl-neuraminic acid, 5-N-Acetyl-9-O-L-lactyl-acetyl-neuraminic acid, 5-N-Acetyl-4-O-acetyl-9-O-lactyl-acetyl-neuraminic acid, 5-N-Acetyl-8-O-methyl-neuraminic acid, 5-N-Acetyl-9-O-acetyl-8-O-methyl-neuraminic acid, 5-N-Acetyl-8-O-sulpho-neuraminic acid, 5-N-Acetyl-9-O-phosphoro-neuraminic acid, 5-N-Acetyl-2-deoxy-2,3-didehydro-neuraminic acid, 5-N-Acetyl-9-O-acetyl-2-deoxy-2,3-didehydro-neuraminic acid, 5-N-Acetyl-2-deoxy-2,3-didehydro-9-O-lactyl-neuraminic acid, 5-N-Acetyl-2,7-anhydro-neuraminic acid, 4-O-Acetyl-5-N-glycolyl-neuraminic acid, 7-O-Acetyl-5-N-glycolyl-neuraminic acid, 8-O-Acetyl-5-N-glycolyl-neuraminic acid, 9-O-Acetyl-5-N-glycolyl-neuraminic acid, 7,9-Di-O-acetyl-5-N-glycolyl-neuraminic acid, 8,9-Di-O-acetyl-5-N-glycolyl-neuraminic acid, 7,8,9-Tri-O-acetyl-5-N-glycolyl-neuraminic acid, 5-N-glycolyl-9-O-lactyl-neuraminic acid, 5-N-glycolyl-8-O-methyl-neuraminic acid, 9-O-Acetyl-5-N-glycolyl-8-O-methyl-neuraminic acid, 7,9-di-O-Acetyl-5-N-glycolyl-8-O-methyl-neuraminic acid, 5-N-glycolyl-8-O-sulpho-neuraminic acid, N-(O-acetyl)glycolylneuraminic acid, N-(O-Methyl)glycolylneuraminic acid, 2-Deoxy-2,3-didehydro-5-N-glycolyl-neuraminic acid, 9-O-Acetyl-2-deoxy-2,3-didehydo-5-N-glycolyl-neuraminic acid, 2-Deoxy-2,3-didehydro-5-N-glycolyl-9-O-lactyl-neuraminic acid, 2-Deoxy-2,3-didehydro-5-N-glycolyl-8-O-methyl-neuraminic acid, 2,7-Anhydro-5-N-glycolyl-neuraminic acid, 2,7-Anhydro-5-N-glycolyl-8-O-methyl-neuraminic acid, 2-Keto-3-deoxynononic acid, 9-O-Acetyl-2-keto-3-deoxynononic acid, and combinations thereof.

In some preferred embodiments, the sialic acid is NANA. In some preferred embodiments, the sialic acid is NGNA. In some preferred embodiments, the sialic acid precursor is N-Acetyl-D-mannosamine. In some preferred embodiments, the sialic acid-containing compound is a ganglioside. In some preferred embodiments, the ganglioside is selected from the group consisting of ganglioside GM1, ganglioside GM2, ganglioside GM3, ganglioside GD1a, ganglioside GD1b, ganglioside GD2, ganglioside GD3, ganglioside GT1b, ganglioside GT3, ganglioside GQ1 and combinations thereof. In some preferred embodiments, the ganglioside is ganglioside GM3.

In some preferred embodiments, the composition is formulated for oral administration and/or administered orally. In some preferred embodiments, the composition is formulated for topical administration and/or administered topically.

In some preferred embodiments, the psoriasis is selected from the group consisting of plaque psoriasis, guttate psoriasis, inverse psoriasis, pustular psoriasis, erythrodermic psoriasis and psoriatic arthritis.

In some preferred embodiments, the sialic acid, sialic acid precursor or sialic acid-containing compound are derived from natural sources. In some preferred embodiments, the natural source is selected from group consisting of a marine animal and marine plant.

In some preferred embodiments, the composition further comprises eicosapentaenoic acid (EPA) and/or docosahexaenoic acid (DHA), wherein the EPA and/or DHA are provided as a free fatty acid, ethyl ester, triglyceride and/or phospholipid.

In some preferred embodiments, the present invention provides oral formulations for treatment or prophylaxis of psoriasis comprising an effective amount of an active compound selected from the group consisting of sialic acid, a sialic acid precursor and a sialic acid containing compound and a pharmaceutically effective carrier.

In some preferred embodiments, the sialic acid is selected from the group consisting of n-glycolylneuraminic acid (NGNA), n-acetylneuraminic acid (NANA), N-Acetyl-D-mannosamine, neuraminic acid, 5-N-Acetyl-4-O-acetyl-neuraminic acid, 5-N-Acetyl-7-O-acetyl-neuraminic acid, 5-N-Acetyl-8-O-acetyl-neuraminic acid, 5-N-Acetyl-9-O-acetyl-neuraminic acid, 5-N-Acetyl-4,9-di-O-acetyl-neuraminic acid, 5-N-Acetyl-7,9-di-O-acetyl-neuraminic acid, 5-N-Acetyl-8,9-di-O-acetyl-neuraminic acid, 5-N-Acetyl-7,8,9-tri-O-acetyl-neuraminic acid, 5-N-Acetyl-9-O-L-lactyl-acetyl-neuraminic acid, 5-N-Acetyl-4-O-acetyl-9-O-lactyl-acetyl-neuraminic acid, 5-N-Acetyl-8-O-methyl-neuraminic acid, 5-N-Acetyl-9-O-acetyl-8-O-methyl-neuraminic acid, 5-N-Acetyl-8-O-sulpho-neuraminic acid, 5-N-Acetyl-9-O-phosphoro-neuraminic acid, 5-N-Acetyl-2-deoxy-2,3-didehydro-neuraminic acid, 5-N-Acetyl-9-O-acetyl-2-deoxy-2,3-didehydro-neuraminic acid, 5-N-Acetyl-2-deoxy-2,3-didehydro-9-O-lactyl-neuraminic acid, 5-N-Acetyl-2,7-anhydro-neuraminic acid, 4-O-Acetyl-5-N-glycolyl-neuraminic acid, 7-O-Acetyl-5-N-glycolyl-neuraminic acid, 8-O-Acetyl-5-N-glycolyl-neuraminic acid, 9-O-Acetyl-5-N-glycolyl-neuraminic acid, 7,9-Di-O-acetyl-5-N-glycolyl-neuraminic acid, 8,9-Di-O-acetyl-5-N-glycolyl-neuraminic acid, 7,8,9-Tri-O-acetyl-5-N-glycolyl-neuraminic acid, 5-N-glycolyl-9-O-lactyl-neuraminic acid, 5-N-glycolyl-8-O-methyl-neuraminic acid, 9-O-Acetyl-5-N-glycolyl-8-O-methyl-neuraminic acid, 7,9-di-O-Acetyl-5-N-glycolyl-8-O-methyl-neuraminic acid, 5-N-glycolyl-8-O-sulpho-neuraminic acid, N-(O-acetyl)glycolylneuraminic acid, N-(O-Methyl)glycolylneuraminic acid, 2-Deoxy-2,3-didehydro-5-N-glycolyl-neuraminic acid, 9-O-Acetyl-2-deoxy-2,3-didehydo-5-N-glycolyl-neuraminic acid, 2-Deoxy-2,3-didehydro-5-N-glycolyl-9-O-lactyl-neuraminic acid, 2-Deoxy-2,3-didehydro-5-N-glycolyl-8-O-methyl-neuraminic acid, 2,7-Anhydro-5-N-glycolyl-neuraminic acid, 2,7-Anhydro-5-N-glycolyl-8-O-methyl-neuraminic acid, 2-Keto-3-deoxynononic acid, 9-O-Acetyl-2-keto-3-deoxynononic acid and combinations thereof.

In some preferred embodiments, the sialic acid is NANA. In some preferred embodiments, the sialic acid is NGNA. In some preferred embodiments, the formulation comprises NGNA and NANA in a ratio of from 1:1 to 100:1. In some preferred embodiments, the sialic acid precursor is N-Acetyl-D-mannosamine. In some preferred embodiments, the sialic acid-containing compound is a ganglioside. In some preferred embodiments, the ganglioside is selected from the group consisting of ganglioside GM1, ganglioside GM2, ganglioside GM3, ganglioside GD1a, ganglioside GD1b, ganglioside GD2, ganglioside GD3, ganglioside GT1b, ganglioside GT3, ganglioside GQ1 and combinations thereof. In some preferred embodiments, the ganglioside is ganglioside GM3. In some preferred embodiments, the formulation comprises at least 2% w/w GM3. In some preferred embodiments, the ratio of GM3 to other gangliosides in the formulation is from 1:2 to 10:1.

In some preferred embodiments, the formulation further comprises eicosapentaenoic acid (EPA) and/or docosahexaenoic acid (DHA), wherein the EPA and/or DHA are provided as a free fatty acid, ethyl ester, triglyceride and/or phospholipid.

In some preferred embodiments, the present invention provides topical formulations for treatment or prophylaxis of psoriasis comprising an effective amount of an active compound selected from the group consisting of sialic acid, a sialic acid precursor and a sialic acid containing compound, a pharmaceutically effective carrier and combinations thereof.

In some preferred embodiments, the sialic acid is selected from the group consisting of n-glycolylneuraminic acid (NGNA), N-Acetyl-D-mannosamine, neuraminic acid, 5-N-Acetyl-4-O-acetyl-neuraminic acid, 5-N-Acetyl-7-O-acetyl-neuraminic acid, 5-N-Acetyl-8-O-acetyl-neuraminic acid, 5-N-Acetyl-9-O-acetyl-neuraminic acid, 5-N-Acetyl-4,9-di-O-acetyl-neuraminic acid, 5-N-Acetyl-7,9-di-O-acetyl-neuraminic acid, 5-N-Acetyl-8,9-di-O-acetyl-neuraminic acid, 5-N-Acetyl-7,8,9-tri-O-acetyl-neuraminic acid, 5-N-Acetyl-9-O-L-lactyl-acetyl-neuraminic acid, 5-N-Acetyl-4-O-acetyl-9-O-lactyl-acetyl-neuraminic acid, 5-N-Acetyl-8-O-methyl-neuraminic acid, 5-N-Acetyl-9-O-acetyl-8-O-methyl-neuraminic acid, 5-N-Acetyl-8-O-sulpho-neuraminic acid, 5-N-Acetyl-9-O-phosphoro-neuraminic acid, 5-N-Acetyl-2-deoxy-2,3-didehydro-neuraminic acid, 5-N-Acetyl-9-O-acetyl-2-deoxy-2,3-didehydro-neuraminic acid, 5-N-Acetyl-2-deoxy-2,3-didehydro-9-O-lactyl-neuraminic acid, 5-N-Acetyl-2,7-anhydro-neuraminic acid, 4-O-Acetyl-5-N-glycolyl-neuraminic acid, 7-O-Acetyl-5-N-glycolyl-neuraminic acid, 8-O-Acetyl-5-N-glycolyl-neuraminic acid, 9-O-Acetyl-5-N-glycolyl-neuraminic acid, 7,9-Di-O-acetyl-S5N-glycolyl-neuraminic acid, 8,9-Di-O-acetyl-5-N-glycolyl-neuraminic acid, 7,8,9-Tri-O-acetyl-5-N-glycolyl-neuraminic acid, 5-N-glycolyl-9-O-lactyl-neuraminic acid, 5-N-glycolyl-8-O-methyl-neuraminic acid, 9-O-Acetyl-5-N-glycolyl-8-O-methyl-neuraminic acid, 7,9-di-O-Acetyl-5-N-glycolyl-8-O-methyl-neuraminic acid, 5-N-glycolyl-8-O-sulpho-neuraminic acid, N-(O-acetyl)glycolylneuraminic acid, N-(O-Methyl)glycolylneuraminic acid, 2-Deoxy-2,3-didehydro-5-N-glycolyl-neuraminic acid, 9-O-Acetyl-2-deoxy-2,3-didehydo-5-N-glycolyl-neuraminic acid, 2-Deoxy-2,3-didehydro-5-N-glycolyl-9-O-lactyl-neuraminic acid, 2-Deoxy-2,3-didehydro-5-N-glycolyl-8-O-methyl-neuraminic acid, 2,7-Anhydro-5-N-glycolyl-neuraminic acid, 2,7-Anhydro-5-N-glycolyl-8-O-methyl-neuraminic acid, 2-Keto-3-deoxynononic acid, 9-O-Acetyl-2-keto-3-deoxynononic acid and combinations thereof.

In some preferred embodiments, the sialic acid is NGNA. In some preferred embodiments, the formulation comprises NGNA in a concentration of at least 2% wt/wt of the composition. In some preferred embodiments, the sialic acid precursor is N-Acetyl-D-mannosamine. In some preferred embodiments, the sialic acid-containing compound is a ganglioside. In some preferred embodiments, the ganglioside is selected from the group consisting of ganglioside GM1, ganglioside GM2, ganglioside GM3, ganglioside GD1a, ganglioside GD1b, ganglioside GD2, ganglioside GD3, ganglioside GT1b, ganglioside GT3, ganglioside GQ1 and combinations thereof. In some preferred embodiments, the ganglioside is ganglioside GM3. In some preferred embodiments, the formulation comprises at least 2% w/w GM3. In some preferred embodiments, the ratio of GM3 to other gangliosides in the formulation is from 1:2 to 10:1. In some preferred embodiments, the formulation further comprises eicosapentaenoic acid (EPA) and/or docosahexaenoic acid (DHA), wherein the EPA and/or DHA are provided as a free fatty acid, ethyl ester, triglyceride and/or phospholipid.

### Definitions

As used herein, “sialic acid” is a generic term for the N- or O-substituted derivatives of neuraminic acid, a monosaccharide with a nine-carbon backbone. It is also the name for the most common member of this group, N-acetylneuraminic acid (Neu5Ac or NANA). Sialic acids are found widely distributed in animal tissues and to a lesser extent in other species ranging from plants and fungi to yeasts and bacteria, mostly in glycoproteins and gangliosides. The amino group generally bears either an acetyl or glycolyl group but other modifications have been described. The hydroxyl substituents may vary considerably: acetyl, lactyl, methyl, sulfate, and phosphate groups have been found.

As used herein, the term “sialic acid precursor” refers to a compound that is used in the biosynthesis of a sialic acid.

As used herein, the term “extract” refers to a substance made by extracting a part of a raw material.

As used herein, the term “phytonutrient” refers to organic compounds isolated from plants that have a biological effect, and includes, but is not limited to, compounds of the following classes: isoflavonoids, oligomeric proanthcyanidins, indol-3-carbinol, sulforaphone, fibrous ligands, plant phytosterols, ferulic acid, anthocyanocides, triterpenes, omega 3/6 fatty acids, polyacetylene, quinones, terpenes, cathechins, gallates, and quercitin.

As used herein, the terms “nutraceutical agent,” and related terms, refer to natural, bioactive chemical compounds that have health promoting, disease preventing or medicinal properties. As used herein, the terms “subject” and “patient” refer to any animal, such as a mammal (e.g., human, horse, dog, cat, bovine, swine, and sheep), bird, or fish. As used herein, the term “physiologically acceptable carrier” refers to any carrier or excipient commonly used with pharmaceuticals. Such carriers or excipients include, but are not limited to, oils, starch, sucrose and lactose.

As used herein, the term “oral delivery vehicle” refers to any means of delivering a pharmaceutical orally, including, but not limited to, capsules, pills, tablets and syrups.

As used herein, the term “food product” refers to any food or feed suitable for consumption by humans, non-ruminant animals, or ruminant animals. The “food product” may be a prepared and packaged food (e.g., mayonnaise, salad dressing, bread, or cheese food) or an animal feed (e.g., extruded and pelleted animal feed or coarse mixed feed). “Prepared food product” means any pre-packaged food approved for human consumption.

As used herein, the term “foodstuff” refers to any substance fit for human or animal consumption.

As used herein, the term “dietary supplement” refers to a small amount of a compound for supplementation of a human or animal diet packaged in single or multiple does units. Dietary supplements do not generally provide significant amounts of calories but may contain other micronutrients (e.g., vitamins or minerals).

As used herein, the term “nutritional supplement” refers to a composition comprising a “dietary supplement” in combination with a source of calories. In some embodiments, nutritional supplements are meal replacements or supplements (e.g., nutrient or energy bars or nutrient beverages or concentrates).

### DETAILED DESCRIPTION OF THE INVENTION

The present invention relates to compositions and methods for treating psoriasis, and in particular to compositions and methods that utilize sialic acids or sialic acid-containing compounds such as gangliosides to treat psoriasis. As demonstrated in the examples, topical and oral formulations containing sialic acid and/or sialic acid containing compounds are effective to treat psoriasis. Types of psoriasis which may treated by the compositions and formulation of the present invention include, but are not limited to, plaque psoriasis, guttate psoriasis, inverse psoriasis, pustular psoriasis, erythrodermic psoriasis, and psoriatic arthritis. In preferred embodiments, an effective amount of sialic acid or sialic acid-containing compounds is administered orally or topically to a subject that has psoriasis. In preferred embodiments, the effective amount is sufficient to reduce symptoms associated with the type of psoriasis being treated. In other embodiments, an effective amount of sialic acid or sialic acid-containing compounds is administered orally or topically to a subject at risk of developing psoriasis to provide prophylaxis.

The use of a variety of sialic acids and sialic acid-containing compounds is contemplated. Examples include, but are not limited to, N- or O-substituted derivatives of neuraminic acid and precursors of sialic acids, including, but not limited to n-glycolylneuraminic acid (NGNA), n-acetylneuraminic acid (NANA), N-Acetyl-D-mannosamine, neuraminic acid, 5-N-Acetyl-4-O-acetyl-neuraminic acid, 5-N-Acetyl-7-O-acetyl-neuraminic acid, 5-N-Acetyl-8-O-acetyl-neuraminic acid, 5-N-Acetyl-9-O-acetyl-neuraminic acid, 5-N-Acetyl-4,9-di-O-acetyl-neuraminic acid, 5-N-Acetyl-7,9-di-O-acetyl-neuraminic acid, 5-N-Acetyl-8,9-di-O-acetyl-neuraminic acid, 5-N-Acetyl-7,8,9-tri-O-acetyl-neuraminic acid, 5-N-Acetyl-9-O-L-lactyl-acetyl-neuraminic acid, 5-N-Acetyl-4-O-acetyl-9-O-lactyl-acetyl-neuraminic acid, 5-N-Acetyl-8-O-methyl-neuraminic acid, 5-N-Acetyl-9-O-acetyl-8-O-methyl-neuraminic acid, 5-N-Acetyl-8-O-sulpho-neuraminic acid, 5-N-Acetyl-9-O-phosphoro-neuraminic acid, 5-N-Acetyl-2-deoxy-2,3-didehydro-neuraminic acid, 5-N-Acetyl-9-O-acetyl-2-deoxy-2,3-didehydro-neuraminic acid, 5-N-Acetyl-2-deoxy-2,3-didehydro-9-O-lactyl-neuraminic acid, 5-N-Acetyl-2,7-anhydro-neuraminic acid, 4-O-Acetyl-5-N-glycolyl-neuraminic acid, 7-O-Acetyl-5-N-glycolyl-neuraminic acid, 8-O-Acetyl-5-N-glycolyl-neuraminic acid, 9-O-Acetyl-5-N-glycolyl-neuraminic acid, 7,9-Di-O-acetyl-5-N-glycolyl-neuraminic acid, 8,9-Di-O-acetyl-5-N-glycolyl-neuraminic acid, 7,8,9-Tri-O-acetyl-5-N-glycolyl-neuraminic acid, 5-N-glycolyl-9-O-lactyl-neuraminic acid, 5-N-glycolyl-8-O-methyl-neuraminic acid, 9-O-Acetyl-5-N-glycolyl-8-O-methyl-neuraminic acid, 7,9-di-O-Acetyl-5-N-glycolyl-8-O-methyl-neuraminic acid, 5-N-glycolyl-8-O-sulpho-neuraminic acid, N-(O-acetyl)glycolylneuraminic acid, N-(O-Methyl)glycolylneuraminic acid, 2-Deoxy-2,3-didehydro-5-N-glycolyl-neuraminic acid, 9-O-Acetyl-2-deoxy-2,3-didehydo-5-N-glycolyl-neuraminic acid, 2-Deoxy-2,3-didehydro-5-N-glycolyl-9-O-lactyl-neuraminic acid, 2-Deoxy-2,3-didehydro-5-N-glycolyl-8-O-methyl-neuraminic acid, 2,7-Anhydro-5-N-glycolyl-neuraminic acid, 2,7-Anhydro-5-N-glycolyl-8-O-methyl-neuraminic acid, 2-Keto-3-deoxynononic acid, and 9-O-Acetyl-2-keto-3-deoxynononic acid. In some particularly preferred embodiments, the sialic acid is n-glycolylneuraminic acid (NGNA) and/or n-acetylneuraminic acid (NANA) or the precursor N-Acetyl-D-mannosamine.

In some preferred embodiments, the sialic acid or sialic acid precursor have a purity selected from the group consisting of greater than 90%, 95%, 99%, and 99.5% pure. The use of sialic acids and precursors from a variety sources is contemplated. In some embodiments, the sialic acid or sialic acid precursor are derived from natural sources. In some embodiments, the natural source is a non-animal source. In some embodiments, the sialic acid or sialic acid precursor are chemically synthesized.

In some embodiments, NGNA is provided from sea cucumbers, e.g., an extract of sea cucumbers, or is prepared from chitin. In some embodiments, the NGNA is preferably about greater than 90%, 95%, 99% or 99.9% pure. In some embodiments, the NGNA is HPLC purified. In some embodiments, NGNA is prepared as described in WO 00/38967, incorporated by reference herein its entirety. For example, N-glycolylneuraminic acid can be purchased commercially from, for example, Sigma Chemical Company, St. Louis, Mo. N-glycolylneuraminic acid also can be synthesized. For example, CMP-N-acetylneuraminic acid hydroxylase can be used to synthesize N-glycolylneuraminic acid as its CMP-glycoside. See, Schlenzka et al., Glycobiolog, 1994, 4(5):675-683. Non-enzymatic methods of synthesis include, for example, synthesis from N-acetylneuraminic acid using methanol or hydrochloric acid and benzylalcohol. Other synthesis methods are described in Choi et al., J. Org. Chem., 1996, 61:8/39 (from mannosamine), Faillard et al., J. Physiol. Chem.′ 1965, 344:167 (from glucosamine), U.S. Pat. No. 4,774,326 and U.S. Pat. No.4,774,327, both of which are incorporated by reference herein in their entirety.

In some embodiments, NANA in may be obtained from commercial suppliers such as Jennewein Biotechnologie GmbH, Rheinbreitbach, Germany.

In other preferred embodiments, sialic acid-containing compounds are utilized. Suitable sialic acid-containing compounds include, but are not limited to, gangliosides. Gangliosides are molecules composed of a glycosphingolipid (ceramide and oligosaccharide) with one or more sialic acids (e.g. n-acetylneuraminic acid, NANA) linked on the sugar chain. Suitable gangliosides include those with 1, 2, 3, or 4 attached sialic acids. In some preferred embodiments, gangliosides are provided from natural sources. In these embodiments, the natural sources are preferably fractioned to provide fractions enriched for gangliosides. Suitable fractionation methods include but are not limited to column chromatography, super critical fluid extraction, moving bed chromatography, and similar methods. Preferred natural sources include but are not limited to fish roe (e.g., herring or salmon roe), whey, colostrum and avian eggs. In some preferred embodiments, the ganglioside is one or more of the gangliosides in Table 1.

Gangliosides

| | | |
|---|---|---|
| GM1 | monosialotetrahexosylganglioside | bDGalp(1-3)bDGalNAc[aNeu5Ac(2- |
| | | 3)]bDGalp(1-4)bDGlcp(1-1)Cer |
| GM2 | | N-Acetyl-D-galactose-beta-1,4-[N- |
| | | Acetylneuraminidate- alpha-2,3-]- |
| | | Galactose-beta-1,4-glucose-alpha- |
| | | ceramide |
| GM3 | Monosialodihexosylganglioslde | aNeu5Ac(2-3)bDGalp(1-4)bDGlcp(1- |
| | NANA-Gal-Glc-ceramide | 1)Cer |
| GD1a | | aNeu5Ac(2-3)bDGalp(1- |
| | | 3)bDGalNAc(1-4)[aNeu5Ac(2- |
| | | 3)]bDGalp(1-4)bDGlcp(1-1)Cer |
| GD1b | | bDGalp(1-3)bDGalNAc(1- |
| | | 4)[aNeu5Ac(2-8)aNeu5Ac(2- |
| | | 3)]bDGalp(1-4)bDGlcp(1-1)Cer |
| GD2 | | bDGalpNAc(1-4)[aNeu5Ac(2- |
| | | 8)aNeu5Ac(2-3)]bDGalp(1-4)bDGlcp(1- |
| | | 1)Cer |
| GD3 | | aNeu5Ac(2-8)aNeu5Ac(2-3)bDGalp(1- |
| | | 4)bDGlcp(1-1)Cer |
| GT1b | | aNeu5Ac(2-3)bDGalp(1- |
| | | 3)bDGalNAc(1-4)[aNeu5Ac(2- |
| | | 8)aNeu5Ac(2-3)]bDGalp(1-4)bDGlcp(1- |
| | | 1)Cer |
| GT3 | | aNeu5Ac(2-8)aNeu5Ac(2- |
| | | 8)aNeu5Ac(2-3)bDGal(1-4)bDGlc(1- |
| | | 1)Cer |
| GQ1 | | aNeu5Ac(2-8)aNeu5Ac(2-3)bDGalp(1- |
| | | 3)bDGalNAc(1-4)[aNeu5Ac(2- |
| | | 8)aNeu5Ac(2-3)]bDGalp(1-4)bDGlcp(1- |
| | | 1)Cer |

where
aNeu5Ac = N-acetyl-alpha-neuraminic acid
aNeu5Ac9Ac = N-acetyl-9-O-acetylneuraminic acid
bDGalp = beta-D-galactopyranose
bDGalpNAc = N-acetyl-beta-D-galactopyranose
bDGlcp = beta-D-glucopyranose
Cer = ceramide (general N-acylated sphingoid)

In some embodiments, compositions comprising a combination of sialic acids and sialic acid precursors are provided. In some embodiments, the compositions comprise at least two isolated and purified sialic acids or sialic acid precursors. In some embodiments, the at least two sialic acids or sialic acid precursors have a purity selected from the group consisting of greater than 90%, 95%, 99%, and 99.5% pure. In some embodiments, the at least two sialic acids or sialic acid precursors are selected from the group consisting of n-glycolylneuraminic acid, n-acetylneuraminic acid and N-Acetyl-D-mannosamine. In some embodiments, the composition may comprise two or more of the following sialic acids: Neuraminic acid, 5-N-Acetyl-4-O-acetyl-neuraminic acid, 5-N-Acetyl-7-O-acetyl-neuraminic acid, 5-N-Acetyl-8-O-acetyl-neuraminic acid, 5-N-Acetyl-9-O-acetyl-neuraminic acid, 5-N-Acetyl-4,9-di-O-acetyl-neuraminic acid, 5-N-Acetyl-7,9-di-O-acetyl-neuraminic acid, 5-N-Acetyl-8,9-di-O-acetyl-neuraminic acid, 5-N-Acetyl-7,8,9-tri-O-acetyl-neuraminic acid, 5-N-Acetyl-9-O-L-lactyl-acetyl-neuraminic acid, 5-N-Acetyl-4-O-acetyl-9-O-lactyl-acetyl-neuraminic acid, 5-N-Acetyl-8-O-methyl-neuraminic acid, 5-N-Acetyl-9-O-acetyl-8-O-methyl-neuraminic acid, 5-N-Acetyl-8-O-sulpho-neuraminic acid, 5-N-Acetyl-9-O-phosphoro-neuraminic acid, 5-N-Acetyl-2-deoxy-2,3-didehydro-neuraminic acid, 5-N-Acetyl-9-O-acetyl-2-deoxy-2,3-didehydro-neuraminic acid, 5-N-Acetyl-2-deoxy-2,3-didehydro-9-O-lactyl-neuraminic acid, 5-N-Acetyl-2,7-anhydro-neuraminic acid, 4-O-Acetyl-5-N-glycolyl-neuraminic acid, 7-O-Acetyl-5-N-glycolyl-neuraminic acid, 8-O-Acetyl-5-N-glycolyl-neuraminic acid, 9-O-Acetyl-5-N-glycolyl-neuraminic acid, 7,9-Di-O-acetyl-5-N-glycolyl-neuraminic acid, 8,9-Di-O-acetyl-5-N-glycolyl-neuraminic acid, 7,8,9-Tri-O-acetyl-5-N-glycolyl-neuraminic acid, 5-N-glycolyl-9-O-lactyl-neuraminic acid, 5-N-glycolyl-8-O-methyl-neuraminic acid, 9-O-Acetyl-5-N-glycolyl-8-O-methyl-neuraminic acid, 7,9-di-O-Acetyl-5-N-glycolyl-8-O-methyl-neuraminic acid, 5-N-glycolyl-8-O-sulpho-neuraminic acid, N-(O-acetyl)glycolylneuraminic acid, N-(O-Methyl)glycolylneuraminic acid, 2-Deoxy-2,3-didehydro-5-N-glycolyl-neuraminic acid, 9-O-Acetyl-2-deoxy-2,3-didehydo-5-N-glycolyl-neuraminic acid, 2-Deoxy-2,3-didehydro-5-N-glycolyl-9-O-lactyl-neuraminic acid, 2-Deoxy-2,3-didehydro-5-N-glycolyl-8-O-methyl-neuraminic acid, 2,7-Anhydro-5-N-glycolyl-neuraminic acid, 2,7-Anhydro-5-N-glycolyl-8-O-methyl-neuraminic acid, 2-Keto-3-deoxynononic acid, and 9-O-Acetyl-2-keto-3-deoxynononic acid. In some embodiments, the composition may comprise three or more sialic acids or precursors.

In some embodiments, the first sialic acid or precursor and second sialic acid or precursor are provided at a ratio of about 20:1 to 1:20, 10:1 to 1:10, 5:1 to 1:5, 2:1 to 1:2, 20:1 to 10:1, 10:1 to 5:1, 5:1 to 2:1, 2:1 to 1:1. In some embodiments, the first sialic acid or precursor may be one of n-glycolylneuraminic acid, n-acetylneuraminic acid and N-Acetyl-D-mannosamine and the second sialic acid or precursor may be another of n-glycolylneuraminic acid, n-acetylneuraminic acid and N-Acetyl-D-mannosamine. In some embodiments, the compositions comprise NGNA and NANA in a ratio of from 1:1 to 1000:1, 1:1 to 100:1, 1:1 to 20:1, 1:1 to 10:1, 1:1 to 5:1, and 1:1 to 2:1. In some embodiments, the compositions comprise NANA and NGNA in a ratio of from 1:1 to 1000:1, 1:1 to 100:1, 1:1 to 20:1, 1:1 to 10:1, 1:1 to 5:1, and 1:1 to 2:1.

In some embodiments, the composition preferably comprise the gangliosides GM1, GM2 and/or GM3. In some preferred embodiments, the concentration of gangliosides in a composition of the present invention, wt/wt of the composition expressed as a percentage, is from 0.1% to 100% wt/wt, 0.5% to 100% wt/wt, 1.0% to 100% wt/wt, 5% to 100% wt/wt, 10% to 100% wt/wt, 20% to 100% wt/wt, 50% to 100% wt/wt, 0.1% to 10% w/w, 1% to 10% w/w, 5% to 10% w/w, 10% to 20% w/w, 20% to 40% w/w or 30% to 50% w/w. In some preferred embodiments, the ratio of GM3 to other gangliosides (e.g., all other gangliosides but GMS such as GM1 and GM2 in the composition is from 1:10 to 10:1, 1:5 to 10:1, 1:2 to 10:1, 1:1 to 10:1, 2:1 to 10:1, 1:2 to 3:1, or 1:2 to 5:1.

In some embodiments, the present invention provides compositions or formulations that are formulated to provide a daily dosage of the sialic acid, sialic acid precursor, or sialic acid-containing compounds (or combinations thereof) selected from the group consisting of 1 mg to 20 g/day, 100 mg to 20 g/day, and 200 mg to 10 g/day. In some embodiments, an effective amount comprises the daily dosages just described.

In some preferred embodiments, the compositions and formulation of the invention further comprise omega-3 compounds. Omega-3 compounds, preferably containing eicosapentaenoic acid (EPA) or docosahexaenoic acid (DHA), may be provided as free fatty acids or more preferably in esterified form as triglycerides, phospholipids, ethyl esters, or combinations thereof. Suitable natural sources of omega-3 compounds include lipid extracts or oils from marine animals and plants including herring, herring roe, other fish such as salmon and anchovy, krill, algae, shrimp, and the like. The omega-3 compounds may preferably be combined or co-extracted with the sialic acids or sialic acid-containing compounds described above.

The compositions of the present invention may be provided in a variety of formulations. In preferred embodiments, the formulations provide the preferred daily dosage of the sialic acid, precursor or sialic acid-containing compound (or combinations thereof). In some embodiments, the compositions are formulated for an administration mode selected from the group consisting of enteral administration, parenteral administration, oral administration, subcutaneous administration, intramuscular administration, and intravenous administration.

In some embodiments, the present invention provides oral dosage forms comprising sialic acid compositions as described above. The ingredients of the oral dosage form of this invention are preferably contained in acceptable excipients and/or carriers for oral consumption. The actual form of the carrier, and thus, the oral dosage form itself, is not critical. The carrier may be a liquid, gel, gel cap, capsule, powder, solid tablet (coated or non-coated), tea, or the like. The oral dosage form is preferably in the form of a tablet or capsule such as a hard gelatin capsule. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). Preferred carriers include calcium carbonate, magnesium stearate, maltodextrin, and mixtures thereof. The various ingredients and the excipient and/or carrier are mixed and formed into the desired form using conventional techniques. The tablet or capsule of the present invention may be coated with an enteric coating that dissolves at a pH of about 6.0 to 7.0. A suitable enteric coating that dissolves in the small intestine but not in the stomach is cellulose acetate phthalate. Further details on techniques for formulation for and administration may be found in the latest edition of Remington's Pharmaceutical Sciences (Maack Publishing Co., Easton, Pa.).

In other embodiments, the compositions are provided as a powder or liquid suitable for adding by the consumer to a feed, food, beverage or water. For example, in some embodiments, the dietary supplement can be administered to a subject in the form of a powder, for instance to be used by mixing into water.

In other embodiments, the present invention provides nutritional supplements (e.g., energy bars or meal replacement bars or beverages) comprising the foregoing compositions. The nutritional supplement may serve as meal or snack replacement and generally provide nutrient calories. Preferably, the nutritional supplements provide carbohydrates, proteins, and fats in balanced amounts. The nutritional supplement can further comprise carbohydrate, simple, medium chain length, or polysaccharides, or a combination thereof. A simple sugar can be chosen for desirable organoleptic properties. Uncooked cornstarch is one example of a complex carbohydrate. If it is desired that it should maintain its high molecular weight structure, it should be included only in food formulations or portions thereof which are not cooked or heat processed since the heat will break down the complex carbohydrate into simple carbohydrates, wherein simple carbohydrates are mono- or disaccharides. The nutritional supplement contains, in one embodiment, combinations of sources of carbohydrate of three levels of chain length (simple, medium and complex; e.g., sucrose, maltodextrins, and uncooked cornstarch).

Sources of protein to be incorporated into the nutritional supplement of the invention can be any suitable protein utilized in nutritional formulations and can include whey protein, whey protein concentrate, whey powder, egg, soy flour, soy milk soy protein, soy protein isolate, caseinate (e.g., sodium caseinate, sodium calcium caseinate, calcium caseinate, potassium caseinate), animal and vegetable protein and mixtures thereof. When choosing a protein source, the biological value of the protein should be considered first, with the highest biological values being found in caseinate, whey, lactalbumin, egg albumin and whole egg proteins. In a preferred embodiment, the protein is a combination of whey protein concentrate and calcium caseinate. These proteins have high biological value; that is, they have a high proportion of the essential amino acids. See Modern Nutrition in Health and Disease, eighth edition, Lea & Febiger, publishers, 1986, especially Volume 1, pages 30-32.

The nutritional supplement can also contain other ingredients, such as one or a combination of other vitamins, minerals, antioxidants, fiber and other dietary supplements (e.g., protein, amino acids, choline, lecithin, omega-3 fatty acids). Selection of one or several of these ingredients is a matter of formulation, design, consumer preference and end-user. The amounts of these ingredients added to the dietary supplements of this invention are readily known to the skilled artisan. Guidance to such amounts can be provided by the U.S. RDA doses for children and adults. Further vitamins and minerals that can be added include, but are not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D₃; cyanocobalamin; sodium selenite; copper sulfate; vitamin A; vitamin C; inositol; potassium iodide.

Flavors, coloring agents, spices, nuts and the like can be incorporated into the product. Flavorings can be in the form of flavored extracts, volatile oils, chocolate flavorings, peanut butter flavoring, cookie crumbs, crisp rice, vanilla or any commercially available flavoring. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee. In one embodiment, the dietary supplement contains cocoa or chocolate.

Emulsifiers may be added for stability of the final product. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product.

Preservatives may also be added to the nutritional supplement to extend product shelf life. Preferably, preservatives such as potassium sorbate, sodium sorbate, potassium benzoate, sodium benzoate or calcium disodium EDTA are used.

In addition to the carbohydrates described above, the nutritional supplement can contain natural or artificial (preferably low calorie) sweeteners, e.g., saccharides, cyclamates, aspartamine, aspartame, acesulfame K, and/or sorbitol. Such artificial sweeteners can be desirable if the nutritional supplement is intended to be consumed by an overweight or obese individual, or an individual with type II diabetes who is prone to hyperglycemia.

The nutritional supplement can be provided in a variety of forms, and by a variety of production methods. In a preferred embodiment, to manufacture a food bar, the liquid ingredients are cooked; the dry ingredients are added with the liquid ingredients in a mixer and mixed until the dough phase is reached; the dough is put into an extruder, and extruded; the extruded dough is cut into appropriate lengths; and the product is cooled. The bars may contain other nutrients and fillers to enhance taste, in addition to the ingredients specifically listed herein.

In still further embodiments, the present invention provides functional foods, including food products, prepared food products, or foodstuffs comprising nutraceutical agents. For example, in some embodiments, beverages and solid or semi-solid foods comprising nutraceutical agents are provided. These forms can include, but are not limited to, beverages (e.g., water, soft drinks, milk and other dairy drinks, and diet drinks), baked goods, puddings, dairy products, confections, snack foods, or frozen confections or novelties (e.g., ice cream, milk shakes), prepared frozen meals, candy, snack products (e.g., chips), soups, spreads, sauces, salad dressings, prepared meat products, cheese, yogurt and any other fat or oil containing foods, and food ingredients (e.g., wheat flour).

In some embodiments, the active agents described above are formulated for topical delivery. General formulations for topical delivery are described in Remington's Pharmaceutical Sciences, 18th Edition, Mack Publishing, p. 1288-1300 [1990]. In accordance with the compositions and method of the present invention, the active agents of the present invention may be administered in the form of a pharmaceutical composition additionally comprising a pharmaceutically acceptable carrier. One skilled in the art will appreciate that suitable methods of administering the extract compositions to an animal, such as a mammal, are available and, although more than one method can be used to administer a particular composition, a particular method and dosage can provide a more immediate and more effective reaction than others. Pharmaceutically acceptable carriers are also well known to those skilled in the art. The choice of carrier will be determined, in part, both by the particular composition and by the particular method used to administer the composition. Accordingly, there is a wide variety of suitable formulations of the pharmaceutical compositions of the present invention.

In some preferred embodiments, the formulations of this invention are designed for topical administration. Typical of such formulations are sprays, ointments, creams, and gels.

Ointments generally are prepared using either (1) an oleaginous base, i.e., one consisting of fixed oils or hydrocarbons, such as white petrolatum or mineral oil, or (2) an absorbent base, i.e., one consisting of an anhydrous substance or substances which can absorb water, for example, anhydrous lanolin. Customarily, following formation of the base, whether oleaginous or absorbent, the active ingredient (e.g., salmon egg extract) is added in an amount affording the desired concentration.

Creams are oil/water emulsions. They consist of an oil phase (internal phase), comprising typically fixed oils, hydrocarbons, and the like, such as waxes, petrolatum, mineral oil, and the like, and an aqueous phase (continuous phase), comprising water and any water-soluble substances, such as added salts. The two phases are stabilized by use of an emulsifying agent, for example, a surface active agent, such as sodium lauryl sulfate; hydrophilic colloids, such as acacia colloidal clays, veegum, and the like. Upon formation of the emulsion, the active ingredient (e.g., salmon egg extract) customarily is added in an amount to achieve the desired concentration.

Gels comprise a base selected from an oleaginous base, water, or an emulsion-suspension base, such as described above. To the base is added a gelling agent which forms a matrix in the base, increasing its viscosity. Examples of gelling agents are hydroxypropyl cellulose, acrylic acid polymers, and the like. Customarily, the active ingredient (IGF-II) is added to the formulation at the desired concentration at a point preceding addition of the gelling agent.

Serums may be watery or thicker liquids, often (but not always) clear in color. Serums are water based making them light in consistency. They are easily and quickly absorbed into the skin and provide an excellent way to deliver topical ingredients including Vitamin C, peptides, alpha hydroxy acids, retinols. Serums may be layered under other serums as well as creams or lotions making them a very flexible product to incorporate into your skin care regimen. Serums are tolerated well by all skin types as long as the individual is not sensitive to any of the ingredients. Serums may include glycerol or glycerine. The amount of extract incorporated into the formulation of this invention is not critical; the concentration should only be in a range sufficient to permit ready application of the formulation to the wound area in an amount which will deliver the desired amount of extract.

Sprays preferably include both aerosol and pump spray formulations.

The present invention may be formulated as necessary with additives used commonly in the pharmaceutical sciences, such as surfactants, oils and fats, polyhydric alcohols, lower alcohols, thickening agents, UV absorbents, light scattering agents, preservatives, antioxidants, antibiotics, chelating agents, pH regulators, flavoring agents, pigments and water.

Examples of surfactants include polyoxyethylene (hereinafter abbreviated as POE-branched alkyl ethers such as POE-octyldodecyl alcohol and POE-2-decyltetradecyl alcohol, POE-alkyl ethers such as POE-oleyl alcohol ether and POE-cetyl alcohol ether, sorbitan esters such as sorbitan monooleate, sorbitan monoisostearate and sorbitan monolaurate, POE-sorbitan esters such as POE-sorbitan monooleate, POE-sorbitan monoisostearate and POE-sorbitan monolaurate, fatty acid esters of glycerol such as glyceryl monooleate, glyceryl monostearate and glyceryl monomyristate, POE-fatty acid esters of glycerol such as POE-glyceryl monooleate, POE-glyceryl monostearate and POE-glyceryl monomyristate, POE-dihydrocholesterol ester, POE-hardened castor oil, POE-hardened castor oil fatty acid esters such as POE-hardened castor oil isostearate, POE-alkylaryl ethers such as POE-octylphenol ether, glycerol esters such as glycerol monoisostearate and glycerol monomyristate, POE-glycerol ethers such as POE-glycerol monoisostearate and POE-glycerol monomyristate, polyglycerol fatty acid esters such as diglyceryl monostearate, decaglyceryl decastearate, decaglyceryl decaisostearate and diglyceryl diisostearate and other nonionic surfactants; potassium salts, sodium salts, diethanolamine salts, triethanolamine salts, amino acid salts and other salts of higher fatty acids such as myristic acid, stearic acid, palmitic acid, behenic acid, isostearic acid and oleic acid, the above alkali salts of ether carboxylic acids, salts of N-acylamino acids, N-acylsalconates, higher alkylsulfonates and other anionic surfactants; alkylamine salts, polyamine, aminoalcohol fatty acids, organic silicone resin, alkyl quaternary ammonium salts and other cationic surfactants; and lecithin, betaine derivatives and other amphoteric surfactants.

Examples of oils and fats include vegetable oils and fats such as castor-oil, olive oil, cacao oil, camellia oil, coconut oil, wood wax, jojoba oil, grape seed oil and avocado oil; animal oils and fats such as mink oil and egg yolk oil; waxes such as beeswax, whale wax, lanolin, carnauba wax and candelilla wax; hydrocarbons such as liquid paraffin, squalene, microcrystalline wax, ceresine wax, paraffin wax and vaseline; natural or synthetic fatty acids such as lauric acid, myristic acid, stearic acid, oleic acid, isostearic acid and behenic acid; natural or higher alcohols such as cetanol, stearyl alcohol, hexyldecanol, octyldecanol and lauryl alcohol; and esters such as isopropyl myristate, isopropyl palmitate, octyldodecyl myristate, octyldodecyl oleate and cholesterol oleate.

Examples of polyhydric alcohols include ethylene glycol, polyethylene glycol, propylene glycol, 1,3-butyrene glycol, 1,4-butyrene glycol, dipropylene glycol, glycerol, diglycerol, triglycerol, tetraglycerol and other polyglycerols, glucose, maltose, maltitose, sucrose, fructose, xylitose, sorbitol, maltotriose, threitol and erythritol.

Examples of thickening agents include naturally-occurring high molecular substances such as sodium alginate, xanthene gum, aluminum silicate, quince seed extract, gum tragacanth, starch, collagen and sodium hyaluronate; semi-synthetic high molecular substances such as methyl cellulose, hydroxyethyl cellulose, carboxymethyl cellulose, soluble starch and cationized cellulose; and synthetic high molecular substances such as carboxyvinyl polymer and polyvinyl alcohol.

Examples of UV absorbents include p-aminobenzoic acid, 2-ethoxyethyl p-methoxycinnamate, isopropyl p-methoxycinnamate, butylmethoxybenzoylmethane, glyceryl-mono-2-ethylhexanoyl-di-p-methoxybenzophenone, digalloyl trioleate, 2,2′-dihydroxy-4-methoxybenzophenone, ethyl-4-bishydroxypropylaminobenzoate, 2-ethylhexyl-2-cyano-3,3′-diphenyl acrylate, ethylhexyl p-methoxycinnamate, 2-ethylhexyl salicylate, glyceryl p-aminobenzoate, homomethyl salicylate, methyl o-aminobenzoate, 2-hydroxy-4-methoxybenzophenone, amyl p-dimethylaminobenzoate, 2-phenylbenzoimidazole-5-sulfonic acid and 2-hydroxy-4-methoxybenzophenone-5-sulfonic acid.

Examples of preservatives include benzoates, salicylates, sorbates, dehydroacetates, p-oxybenzoates, 2,4,4′-trichloro-2′-hydroxydiphenyl ether, 3,4,4′-trichlorocarbanilide, benzalkonium chloride, hinokitiol, resorcinol and ethanol.

Examples of antioxidants include tocopherol, ascorbic acid, butylhydroxyanisole, dibutylhydroxytoluene, nordihydroguaiaretic acid and propyl gallate.

Examples of chelating agents include sodium edetate and sodium citrate.

Examples of antibiotics include penicillin, neomycin, cephalothin, potassium permanganate, selenium sulfide, erythromycin, bacitracin, tethacyclin, chloramphenicol, vancomycin, nitrofurantoin, acrisorcin, chlorodontoin, and flucytosine.

Some of these additives function to enhance the efficacy of the composition by increasing the stability or percutaneous absorbability of the essential components of the present invention.

Also, any dosage form is acceptable, whether in solution, emulsion, powder dispersion, or others. Applicability is wide, including fundamental dosage forms such as lotions, emulsions, creams and gels.

In addition to those stated above, suitable vehicles, carriers and adjuvants include water, vaseline, petrolatum, mineral oil, vegetable oil, animal oil, organic and inorganic waxes, polymers such as xanthanes, gelatin, cellulose, collagen, starch, kaolin, carrageenan, gum arabic, synthetic polymers, alcohols, polyols, and the like. The carrier can also include sustained release carrier such as lypizomes, microsponges, microspheres, or microcapsules, aqueous base ointments, water in oil or oil in water emulsions, gels or the like.

In some preferred embodiments where a topical formulation is utilized, the topical formulation comprises less than 5%, 1%, 0.5%, 0.1% NANA wt/wt or is substantially free of NANA.

### EXAMPLES

Example 1. A child with plaque psoriasis was treated with sea cucumber powder containing 250 micrograms NGNA per 400 mg capsule administered orally. Administration of 5 capsules a day caused symptoms associated with the psoriasis including redness, scaling spots, itching, burning and soreness to be reduced as compared to previous treatments.

Example 2. An adult with psoriasis was treated with 5 mg NGNA per day administered sublingually. Administration caused symptoms associated with the psoriasis including redness, scaling spots, itching, burning and soreness to be reduced as compared to previous treatments.

Example 3. An adult with psoriasis was treated with 5 g NGNA per day administered orally. Administration caused symptoms associated with the psoriasis including redness, scaling spots, itching, burning and soreness to be reduced as compared to previous treatments.

Example 4. An adult with psoriasis was treated with NGNA supplied in a hot water bath. Treatment caused symptoms associated with the psoriasis including redness, scaling spots, itching, burning and soreness to be reduced as compared to previous treatments.

Example 5. An adult with psoriasis was treated with a dermal spray solution of NGNA in water. Treatment caused symptoms associated with the psoriasis including redness, scaling spots, itching, burning and soreness to be reduced as compared to previous treatments.

1-26. (canceled)

27. A topical formulation for treatment or prophylaxis of psoriasis comprising an effective amount of an active compound selected from the group consisting of sialic acid, a sialic acid precursor and a sialic acid containing compound, a pharmaceutically effective carrier and combinations thereof.

28. The topical formulation of claim 27, wherein the sialic acid is selected from the group consisting of n-glycolylneuraminic acid (NGNA), N-Acetyl-D-mannosamine, neuraminic acid, 5-N-Acetyl-4-O-acetyl-neuraminic acid, 5-N-Acetyl-7-O-acetyl-neuraminic acid, 5-N-Acetyl-8-O-acetyl-neuraminic acid, 5-N-Acetyl-9-O-acetyl-neuraminic acid, 5-N-Acetyl-4,9-di-O-acetyl-neuraminic acid, 5-N-Acetyl-7,9-di-O-acetyl-neuraminic acid, 5-N-Acetyl-8,9-di-O-acetyl-neuraminic acid, 5-N-Acetyl-7,8,9-tri-O-acetyl-neuraminic acid, 5-N-Acetyl-9-O-L-lactyl-acetyl-neuraminic acid, 5-N-Acetyl-4-O-acetyl-9-O-lactyl-acetyl-neuraminic acid, 5-N-Acetyl-8-O-methyl-neuraminic acid, 5-N-Acetyl-9-O-acetyl-8-O-methyl-neuraminic acid, 5-N-Acetyl-8-O-sulpho-neuraminic acid, 5-N-Acetyl-9-O-phosphoro-neuraminic acid, 5-N-Acetyl-2-deoxy-2,3-didehydro-neuraminic acid, 5-N-Acetyl-9-O-acetyl-2-deoxy-2,3-didehydro-neuraminic acid, 5-N-Acetyl-2-deoxy-2,3-didehydro-9-O-lactyl-neuraminic acid, 5-N-Acetyl-2,7-anhydro-neuraminic acid, 4-O-Acetyl-5-N-glycolyl-neuraminic acid, 7-O-Acetyl-5-N-glycolyl-neuraminic acid, 8-O-Acetyl-5-N-glycolyl-neuraminic acid, 9-O-Acetyl-5-N-glycolyl-neuraminic acid, 7,9-Di-O-acetyl-5-N-glycolyl-neuraminic acid, 8,9-Di-O-acetyl-5-N-glycolyl-neuraminic acid, 7,8,9-Tri-O-acetyl-5-N-glycolyl-neuraminic acid, 5-N-glycolyl-9-O-lactyl-neuraminic acid, 5-N-glycolyl-8-O-methyl-neuraminic acid, 9-O-Acetyl-5-N-glycolyl-8-O-methyl-neuraminic acid, 7,9-di-O-Acetyl-5-N-glycolyl-8-O-methyl-neuraminic acid, 5-N-glycolyl-8-O-sulpho-neuraminic acid, N-(O-acetyl)glycolylneuraminic acid, N-(O-Methyl)glycolylneuraminic acid, 2-Deoxy-2,3-didehydro-5-N-glycolyl-neuraminic acid, 9-O-Acetyl-2-deoxy-2,3-didehydo-5-N-glycolyl-neuraminic acid, 2-Deoxy-2,3-didehydro-5-N-glycolyl-9-O-lactyl-neuraminic acid, 2-Deoxy-2,3-didehydro-5-N-glycolyl-8-O-methyl-neuraminic acid, 2,7-Anhydro-5-N-glycolyl-neuraminic acid, 2,7-Anhydro-5-N-glycolyl-8-O-methyl-neuraminic acid, 2-Keto-3-deoxynononic acid, 9-O-Acetyl-2-keto-3-deoxynononic acid and combinations thereof.

29. The topical formulation of claim 28, wherein the sialic acid is NGNA.

30. The topical formulation of claim 29, wherein the formulation comprises NGNA in a concentration of at least 2% wt/wt of the composition.

31. The topical formulation of claim 27, wherein the sialic acid precursor is N-Acetyl-D-mannosamine.

32. The topical formulation of claim 27, wherein the sialic acid-containing compound is a ganglioside.

33. The topical formulation of claim 32, wherein the ganglioside is selected from the group consisting of ganglioside GM1, ganglioside GM2, ganglioside GM3, ganglioside GD1a, ganglioside GD1b, ganglioside GD2, ganglioside GD3, ganglioside GT1b, ganglioside GT3, ganglioside GQ1 and combinations thereof.

34. The topical formulation of claim 33, wherein the ganglioside is ganglioside GM3.

35. The topical formulation of claim 34, wherein the formulation comprises at least 2% w/w GM3.

36. The topical formulation of claim 35, wherein the ratio of GM3 to other gangliosides in the formulation is from 1:2 to 10:1.

37. The topical formulation of claim 27, wherein the formulation further comprises eicosapentaenoic acid (EPA) and/or docosahexaenoic acid (DHA), wherein the EPA and/or DHA are provided as a free fatty acid, ethyl ester, triglyceride and/or phospholipid.

38. A method of treating or providing prophylaxis of psoriasis comprising administering to a subject in need thereof an effective amount of an active agent selected from the group consisting of a sialic acid, a sialic acid precursor and a sialic acid containing compound.

39. Method of claim 38, wherein the sialic acid is selected from the group consisting of n-glycolylneuraminic acid (NGNA), n-acetylneuraminic acid (NANA), N-Acetyl-D-mannosamine, neuraminic acid, 5-N-Acetyl-4-O-acetyl-neuraminic acid, 5-N-Acetyl-7-O-acetyl-neuraminic acid, 5-N-Acetyl-8-O-acetyl-neuraminic acid, 5-N-Acetyl-9-O-acetyl-neuraminic acid, 5-N-Acetyl-4,9-di-O-acetyl-neuraminic acid, 5-N-Acetyl-7,9-di-O-acetyl-neuraminic acid, 5-N-Acetyl-8,9-di-O-acetyl-neuraminic acid, 5-N-Acetyl-7,8,9-tri-O-acetyl-neuraminic acid, 5-N-Acetyl-9-O-L-lactyl-acetyl-neuraminic acid, 5-N-Acetyl-4-O-acetyl-9-O-lactyl-acetyl-neuraminic acid, 5-N-Acetyl-8-O-methyl-neuraminic acid, 5-N-Acetyl-9-O-acetyl-8-O-methyl-neuraminic acid, 5-N-Acetyl-8-O-sulpho-neuraminic acid, 5-N-Acetyl-9-O-phosphoro-neuraminic acid, 5-N-Acetyl-2-deoxy-2,3-didehydro-neuraminic acid, 5-N-Acetyl-9-O-acetyl-2-deoxy-2,3-didehydro-neuraminic acid, 5-N-Acetyl-2-deoxy-2,3-didehydro-9-O-lactyl-neuraminic acid, 5-N-Acetyl-2,7-anhydro-neuraminic acid, 4-O-Acetyl-5-N-glycolyl-neuraminic acid, 7-O-Acetyl-5-N-glycolyl-neuraminic acid, 8-O-Acetyl-5-N-glycolyl-neuraminic acid, 9-O-Acetyl-5-N-glycolyl-neuraminic acid, 7,9-Di-O-acetyl-5-N-glycolyl-neuraminic acid, 8,9-Di-O-acetyl-5-N-glycolyl-neuraminic acid, 7,8,9-Tri-O-acetyl-5-N-glycolyl-neuraminic acid, 5-N-glycolyl-9-O-lactyl-neuraminic acid, 5-N-glycolyl-8-O-methyl-neuraminic acid, 9-O-Acetyl-5-N-glycolyl-8-O-methyl-neuraminic acid, 7,9-di-O-Acetyl-5-N-glycolyl-8-O-methyl-neuraminic acid, 5-N-glycolyl-8-O-sulpho-neuraminic acid, N-(O-acetyl)glycolylneuraminic acid, N-(O-Methyl)glycolylneuraminic acid, 2-Deoxy-2,3-didehydro-5-N-glycolyl-neuraminic acid, 9-O-Acetyl-2-deoxy-2,3-didehydo-5-N-glycolyl-neuraminic acid, 2-Deoxy-2,3-didehydro-5-N-glycolyl-9-O-lactyl-neuraminic acid, 2-Deoxy-2,3-didehydro-5-N-glycolyl-8-O-methyl-neuraminic acid, 2,7-Anhydro-5-N-glycolyl-neuraminic acid, 2,7-Anhydro-5-N-glycolyl-8-O-methyl-neuraminic acid, 2-Keto-3-deoxynononic acid, 9-O-Acetyl-2-keto-3-deoxynononic acid, and combinations thereof.

40. Method of claim 39, wherein the sialic acid is NANA.

41. Method of claim 39, wherein the sialic acid is NGNA.

42. Method of claim 38, wherein the sialic acid precursor is N-Acetyl-D-mannosamine.

43. Method of claim 38, wherein the sialic acid-containing compound is a ganglioside.

44. Method of claim 43, wherein the ganglioside is selected from the group consisting of ganglioside GM1, ganglioside GM2, ganglioside GM3, ganglioside GD1a, ganglioside GD1b, ganglioside GD2, ganglioside GD3, ganglioside GT1b, ganglioside GT3, ganglioside GQ1 and combinations thereof.

45. Method of claim 44, wherein the ganglioside is ganglioside GM3.

46-47. (canceled)

48. Method of claim 38, wherein the psoriasis is selected from the group consisting of plaque psoriasis, guttate psoriasis, inverse psoriasis, pustular psoriasis, erythrodermic psoriasis and psoriatic arthritis.

49-50. (canceled)

51. Method of claim 38, wherein the composition further comprises eicosapentaenoic acid (EPA) and/or docosahexaenoic acid (DHA), wherein the EPA and/or DHA are provided as a free fatty acid, ethyl ester, triglyceride and/or phospholipid.
